(19) United States
(12) Patent Application Publication
(10) Pub. No.: US 2021/0121088 A1
(43) Pub. Date: Apr. 29, 2021

(54) CRANIAL PLUG INCLUDING AN INTRACRANIAL MONITORING DEVICE

(71) Applicant: Longeviti Neuro Solutions LLC, Hunt Valley, MD (US)

(72) Inventors: Jesse Christopher, Hunt Valley, MD (US); Bradley Rabinovitz, Severna Park, MD (US); Todd Johnson, Chalfont, PA (US)

(21) Appl. No.: 17/078,607

(22) Filed: Oct. 23, 2020

Related U.S. Application Data
(60) Provisional application No. 62/925,428, filed on Oct. 24, 2019.

(51) Int. Cl. A61B 5/03; A61B 5/00; A61B 34/20

(52) U.S. Cl. CPC A61B 5/031; A61B 2034/2065; A61B 34/20; A61B 5/0022

(57) ABSTRACT

A cranial plug includes an intracranial monitoring device having a probe and a cranial plug housing having an intracranial monitoring device recess. The intracranial monitoring device recess is shaped and dimensioned for positioning of an intracranial monitoring device and includes a central access hole shaped and dimensioned for the passage of the probe of the intracranial monitoring device therethrough to a desired position within the brain.

### CROSS REFERENCE TO RELATED APPLICATION

This application claims the benefit of U.S. Provisional Patent Application Ser. No. 62/925,428, entitled “CRANIAL PLUG INCLUDING AN INTRACRANIAL MONITORING DEVICE,” filed Oct. 24, 2019, which is incorporated herein by reference.

### BACKGROUND OF THE INVENTION

### 1. Field of the Invention

The present invention relates to a cranial plug including an intracranial monitoring device.

### 2. Description of the Related Art

Intracranial monitoring of various pathologies has advanced rapidly in recent years. Such monitoring devices are disclosed in U.S. Pat. Nos. 8,337,413 and 9,339,189 and U.S. Patent Application Publication Nos. 2006/0025704, 2008/0161659, 2008/0262319, 2011/0009716, 2013/0123660, 2014/0210637, and 2018/0325386.

With these developments in mind, it is desirable to improve upon current techniques for the placement of intracranial monitoring devices and the present invention provides for improvements in the placement of such intracranial monitoring devices.

### SUMMARY OF THE INVENTION

In an embodiment, a cranial plug includes an intracranial monitoring device having a probe. The cranial plug also includes a cranial plug housing having an intracranial monitoring device recess. The intracranial monitoring device recess is shaped and dimensioned for positioning of an intracranial monitoring device and includes a central access hole shaped and dimensioned for the passage of the probe of the intracranial monitoring device therethrough to a desired position within the brain.

In some embodiments, the intracranial monitoring device is a wireless intracranial monitoring device.

In some embodiments, the cranial plug housing includes a first end, a second end, a first lateral side, a second lateral side, an upper surface, and a lower surface.

In some embodiments, a recessed surface defining the intracranial monitoring device recess is formed along the upper surface of the cranial plug housing.

In some embodiments, the lower surface is provided with a projection shaped and dimensioned to fit within the cranial hole.

In some embodiments, the intracranial monitoring device recess is formed within the upper surface of the cranial plug housing.

In some embodiments, the intracranial monitoring device recess is shaped and dimensioned for receiving the head of the intracranial monitoring device.

In some embodiments, the central access hole extends from the recess to the lower surface of the cranial plug housing.

In some embodiments, the cranial plug housing includes multiple chemically distinct materials.

In some embodiments, the cranial plug housing includes a central body member made of first material and a second material.

In some embodiments, the first material a sonolucent material.

In some embodiments, the first material is PMMA or cubic zirconium.

In some embodiments, the second material is a flexible material.

In some embodiments, the first material is PMMA or cubic zirconium and the second material is porous polyethylene or expanded polytetrafluoroethylene (EPTFE).

In some embodiments, the second material is a flexible material.

In some embodiments, the second material is porous polyethylene or expanded polytetrafluoroethylene (EPTFE).

In another aspect a method for installing a cranial plug is provided, wherein the cranial plug includes an intracranial monitoring device including a probe and a cranial plug housing having an intracranial monitoring device recess. The intracranial monitoring device recess is shaped and dimensioned for positioning of an intracranial monitoring device therein and includes a central access hole shaped and dimensioned for the passage of a probe of the intracranial monitoring device therethrough to a desired position. The method includes generating virtual images of the cranial plug and the intracranial monitoring device upon initiation of a surgical procedure. The method also includes monitoring in real-time, via a computer based guidance system, the movement of the cranial plug housing and the intracranial monitoring device relative to the patient, wherein tracking devices are integrated into or onto the cranial plug housing and the intracranial monitoring device. Finally, the method includes positioning the cranial plug within the patient.

Other advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of a cranial plug with a wireless intracranial monitoring device.

FIG. 2 is a perspective view of the wireless intracranial monitoring device shown in FIG. 1.

FIG. 3 is a perspective view of the cranial plug housing of the cranial plug shown in FIG. 1.

FIGS. 4, 5, and 6 are respectively a top view, a side view, and a bottom view of the cranial plug housing of the cranial plug shown in FIG. 1.

FIGS. 7, 8, and 9 are respectively a perspective view, a top view, and a side view of the cranial plug shown in FIG. 1.

FIG. 10 is a schematic showing the process of implanting the cranial plug in accordance with the present invention.

FIGS. 11, 12, and 13 show various alternate embodiments of the present cranial plug.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

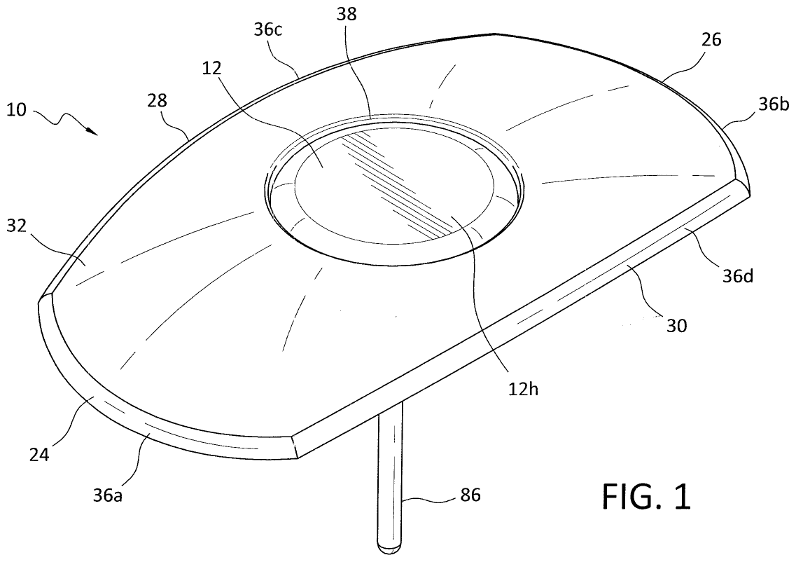

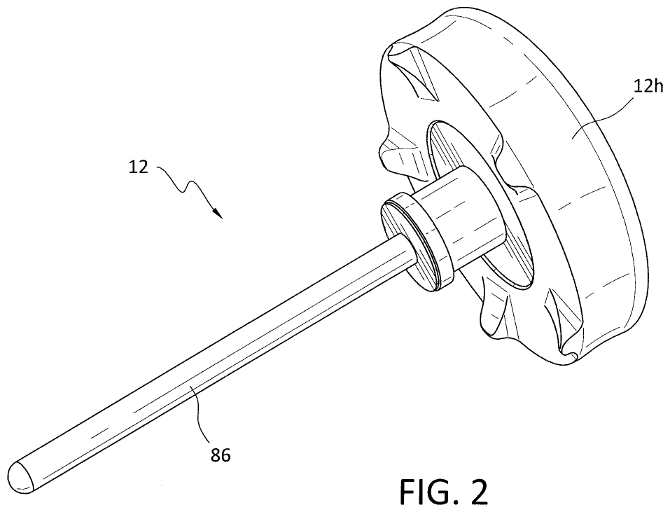

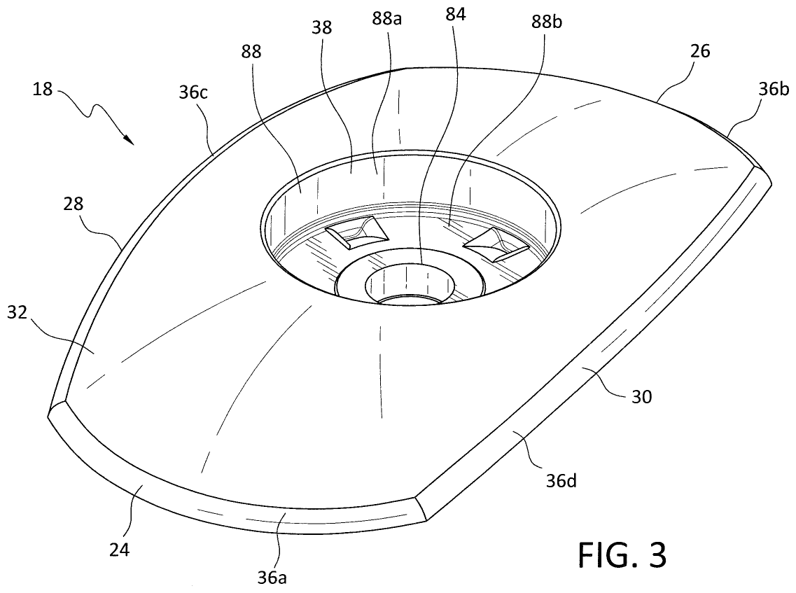

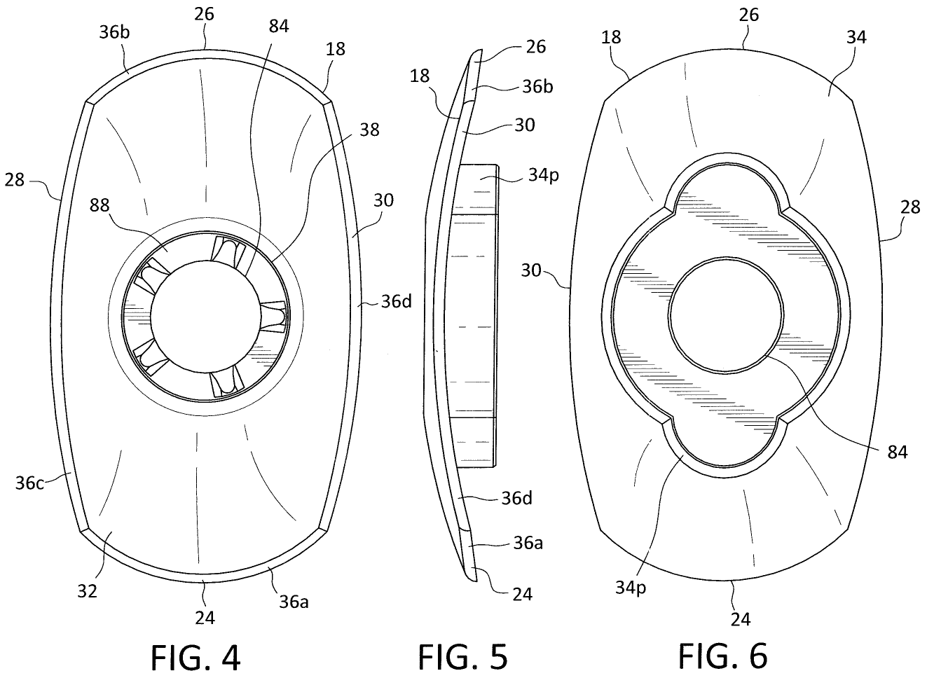

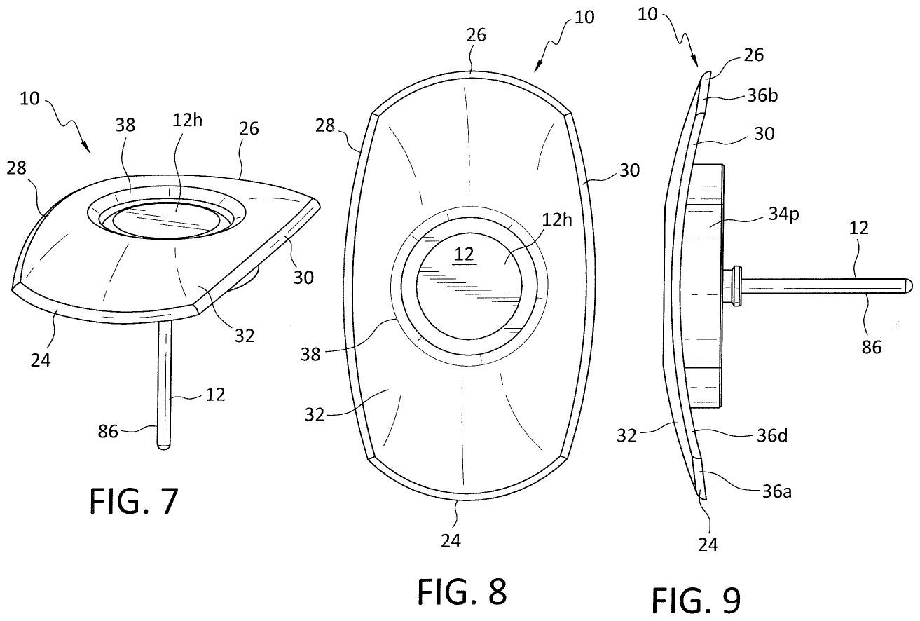

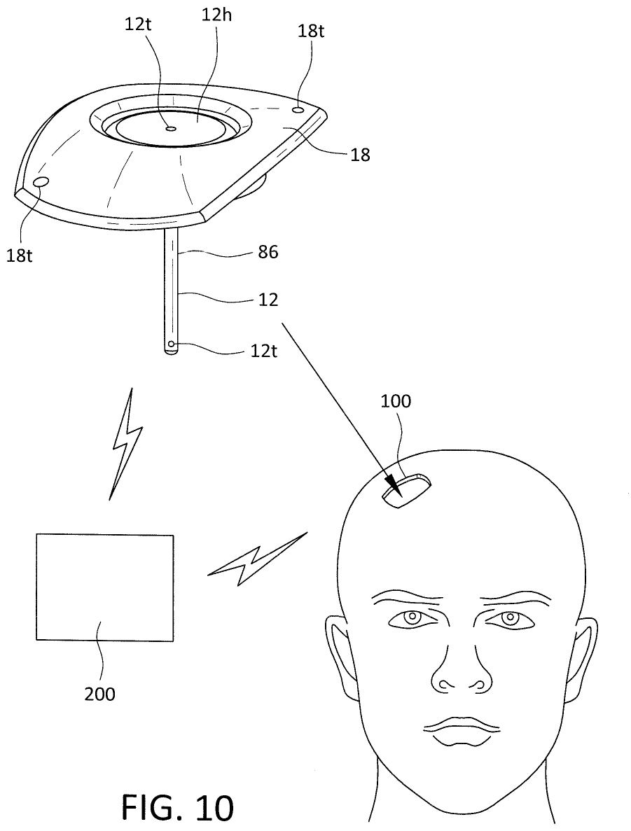

Referring to FIGS. 1 to 10, various embodiments of a cranial plug 10 including a wireless intracranial monitoring device 12 are disclosed. The cranial plug 10 is shaped and dimensioned for positioning within a physician formed cranial hole 100. The cranial plug 10 is further shaped and dimensioned for housing the intracranial monitoring device 12 in a reliable and secure manner. The cranial plug 10 includes a cranial plug housing 18. In accordance with the disclosed embodiment, the intracranial monitoring device 12 will be placed within the cranial plug housing 18, so as to create the cranial plug 10 of the present invention, at the time of surgery.

The cranial plug housing 18 includes a first end 24, a second end 26, a first lateral side 28, and a second lateral side 30. The cranial plug housing 18 also includes an upper surface 32, a lower surface 34, and continuous side walls 36a-d extending between the upper surface 32 and the lower surface 34, as well as about the periphery of the cranial plug housing 18. As will be appreciated based upon the following disclosure, the lower surface 34 is provided with a projection 34p that ultimately fits within the cranial hole 100 to assist in holding the cranial plug 10 in position after installation. In accordance with the disclosed embodiment, the projection 34p is defined by the outer wall of the intracranial monitoring device recess 38 in which the head 12h of the intracranial monitoring device 12 is positioned. With this in mind, the projection 34p is shaped to fit within the cranial hole 100.

While a particular shape of the cranial plug housing 18 in accordance with the disclosed embodiment is disclosed herein for the purpose of explaining the present invention, it is appreciated various shapes may be employed within the spirit of the present invention. As such, the shape of the cranial plug and the mechanism for the creation of the cranial hole are intimately related and may be varied based upon patient needs and requirements.

An intracranial monitoring device recess 38 is formed within the upper surface 32 of the cranial plug housing 18. The intracranial monitoring device recess 38 is shaped and dimensioned for positioning of an intracranial monitoring device 12, in particular, the head 12h of the intracranial monitoring device 12, therein. As such, and as will be appreciated based upon the following disclosure, the intracranial monitoring device recess 38 is provided with a central access hole 84 extending from the recess 38 to the lower surface 34 of the cranial plug housing 18. The central access hole 84 is shaped and dimensioned for the passage of the probe 86 of the wireless intracranial monitoring device 12 therethrough to a desired position within the brain.

The intracranial monitoring device recess 38 in which the wireless intracranial monitoring device 12 is positioned, as well as the central access hole 84 for the passage of the probe 86, is formed within the cranial plug housing 18. The intracranial monitoring device recess 38 is defined by the recessed surface 88 formed along the upper surface 32 of the cranial plug housing 18. In particular, the recessed surface 88 defining the intracranial monitoring device recess 38 is formed along the upper surface 32 of the cranial plug housing 18. In accordance with a disclosed embodiment, the recessed surface 88 is defined by a cylindrical interior wall 88a extending downwardly from the remainder of the upper surface 32 and a base wall 88b, wherein the base wall 88b defines a ledge upon which the intracranial monitoring device 12 sits when positioned within the intracranial monitoring device recess 38.

As briefly discussed above, the intracranial monitoring device recess 38 defined within the cranial plug housing 18 is shaped and dimensioned for placement of the wireless intracranial monitoring device 12 therein. As those skilled in the art will appreciate, and as explained above in conjunction with the prior embodiment, a variety of wireless intracranial monitoring devices are known in the art and the present cranial plug housing 18 may be adapted to accommodate a variety of these wireless intracranial monitoring devices 12. However, and in accordance with a preferred embodiment of the present invention, the wireless intracranial monitoring device 12 is one or a combination of the wireless intracranial pressure monitoring devices disclosed in U.S. Pat. Nos. 8,337,413 and 9,339,189 and U.S. Patent Application Publication Nos. 2006/0025704, 2008/0161659, 2008/0262319, 2011/0009716, 2013/0123660, and 2014/0210637, all of which are incorporated herein by reference. In accordance with this embodiment, the cranial plug housing 18 should have a surface area along its upper surface 32 sufficient to accommodate various wireless intracranial monitoring devices.

Further functionality may be achieved by using a wireless intracranial monitoring device offering multiple sensing capabilities (multimodal), for example, as disclosed in U.S. Patent Application Publication No. 2018/0325386, entitled “MULTIPLE IMPLANTABLE SENSOR PROBE,” published Nov. 15, 2018, which is incorporated herein by reference. While a wireless intracranial monitoring device is disclosed in accordance with preferred embodiments of the present invention, it is appreciated wired intracranial monitoring device and other intracranial monitoring devices could be used without departing from the spirit of the present invention.

It is appreciated the housing of the cranial plug may be further modified to improve positioning and identification thereof through the inclusion of various structural elements as disclosed in Applicant's own U.S. Patent Application Publication No. 2018/0304055, entitled “CEREBRAL SPINAL FLUID SHUNT PLUG,” published Oct. 25, 2019, which is incorporated herein by reference. Briefly, physical bumps, an RFID (radio-frequency identification) device, and/or radiographic and/or acoustic properties may be incorporated into the housing.

Installation of the cranial plug may be completed in a manner similar to that disclosed in U.S. Patent Application Publication No. 2018/0304055, entitled “CEREBRAL SPINAL FLUID SHUNT PLUG,” published Oct. 25, 2019, which is incorporated herein by reference. The procedures described in the '055 publication, however, may be supplemented with positioning technology as described below. As those skilled in the art will appreciate, proper positioning of the probe 86 of the intracranial monitoring device 12 is critical. The orientation of the wireless intracranial monitoring device 12, for example, the probe 86 of the intracranial monitoring device 12, is critical to understand and appreciate so as to avoid eloquent structures on the cortex of the brain; such as the trajectory between Kocher's point and the ventricles or previously necrosed brain damage from a traumatic injury or due to high intracranial pressure. Furthermore, by identifying the relative positions of the wireless intracranial monitoring device 12 and the cranial plug housing 18 to the cortex, the relative positioning of various structures may be surmised and eloquent structures of the brain are avoided. For example, and through the use of the present cranial plug 10 in conjunction with various computer based surgical guidance systems 200 as discussed below, it is possible for surgeons to fully appreciate the relationship of the intracranial monitoring device 12 to the cranial plug 10 and, therefore, the cortex. This enables surgeons to place the intracranial monitoring device 12 in a manner that minimizes the potential for cortical damage.

In practice, and prior to initiating the surgical procedure, virtual images of the cranial plug 10, including the intracranial monitoring device 12, are generated. Virtual images of the patient, including the approximate location of the cranial plug 10 are also generated. Upon initiation of the surgical procedure, movement of the actual cranial plug 10, including the cranial plug housing 18 and the intracranial monitoring device 12, relative to the patient is monitored in real-time. This is achieved by the integration of tracking devices 12t, 18t into or onto the respective cranial plug housing 18 and the intracranial monitoring device 12. Additional tracking devices may be applied to the patient in a manner known to those skilled in the art. It should be appreciated that the tracking devices 12t, 18t may take a variety of forms so long as the computer based guidance system 200 is capable of identifying the real-time movement of the various components of the cranial plug 10 being tracked. For example, the tracking devices may take the form of external tracking devices attached to the cranial plug, tracking devices integrated into the cranial plug, or existing structures of the cranial plug that are readily identifiable via the sensing structure of the computer based guidance system 200. Sensing may be achieved via various known techniques, including, but not limited to, infrared, electromagnetic, optical, or other sensing techniques.

With the virtual images and the tracking devices, when used in conjunction with a computer based surgical guidance system 200, the cranial plug 10 is properly positioned within the patient. Once the wireless intracranial monitoring device 12 is properly positioned, it may be actuated utilizing well known procedures, and the procedure is completed in accordance with known medical procedures.

While cranial plug housing of the various embodiments disclosed above are made of clear PMMA, it is contemplated, the cranial plug housing could be of a multi-material construction with the use of different materials in different elements of the cranial plug housing so as to expand the functionality thereof.

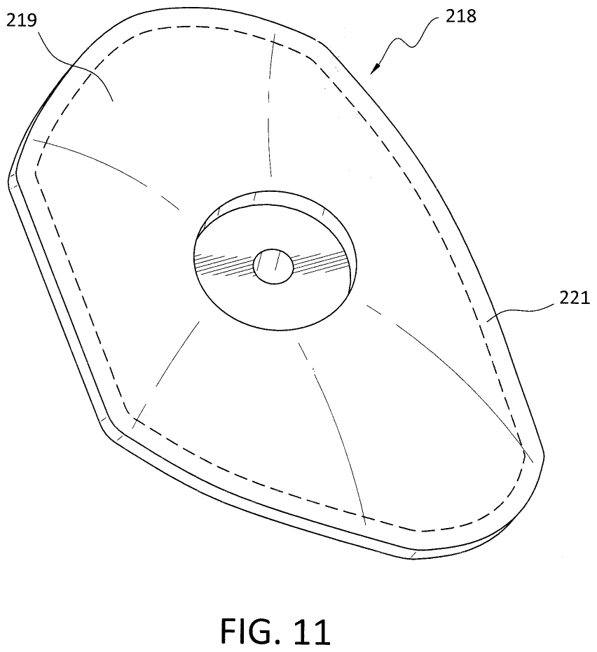

For example, and with reference to FIG. 11, the cranial plug housing 218 includes a central body member 219 made of rigid sonolucent PMMA and a flexible perimeter member 221 made of porous polyethylene. Such a construction provides medical practitioners with a large sonolucent area for transcranioplasty ultrasound as provided by the central body member 219 and a malleable perimeter that optimizes a smooth transition between the implant perimeter and the native skull as provided by the flexible perimeter member 221.

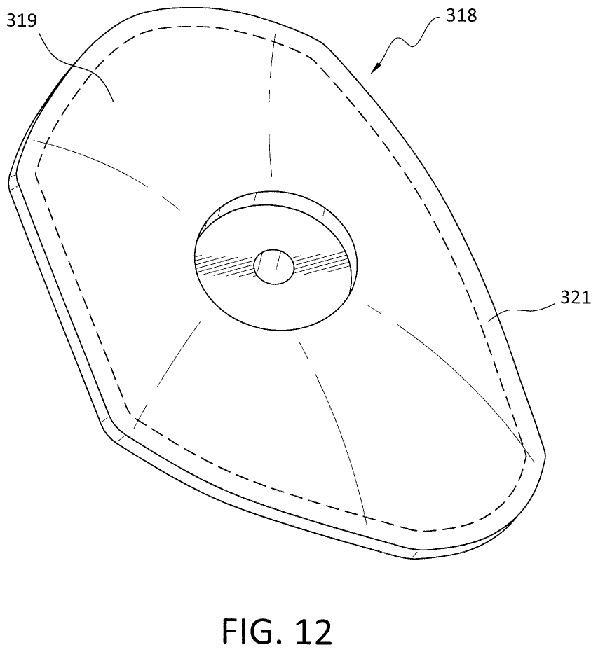

In accordance with another embodiment as shown with reference to FIG. 12, the cranial plug housing 318 includes a central body member 319 made of cubic zirconium (or any other rigid sonolucent material) and a flexible perimeter member 321 made of expanded polytetrafluoroethylene (EPTFE), silicon, or other malleable material.

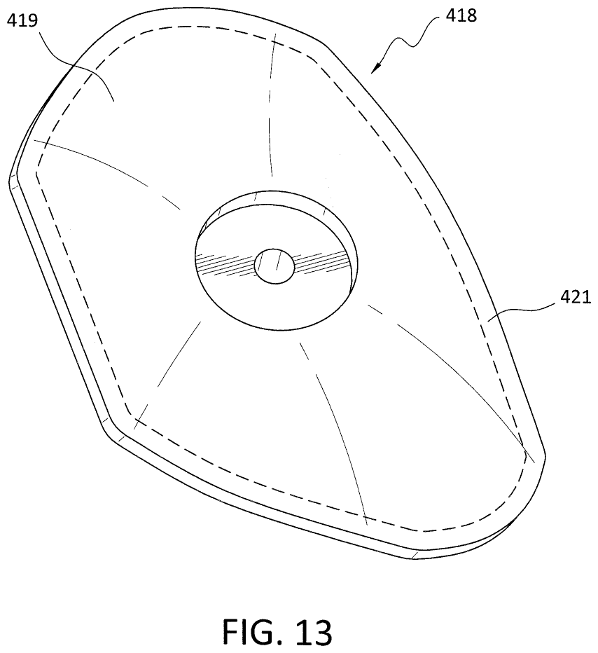

In accordance with yet another embodiment, as shown with reference to FIG. 13, the cranial plug housing 418 could be different compositions of the same material, with the different compositions being selected to enhance sonolucency and aesthetic fixation. For example, the central body member 419 of the cranial plug housing 418 could be rigid sonolucent PMMA while the flexible perimeter member 421 of the cranial plug housing could be PMMA with elastomer additives that change its material properties from rigid to malleable. In this way, the implant could have an optimal smooth transition from the perimeter of the implant to the native skull.

While the preferred embodiments have been shown and described, it will be understood that there is no intent to limit the invention by such disclosure, but rather, the disclosure is intended to cover all modifications and alternate constructions falling within the spirit and scope of the invention.

1. A cranial plug, comprising:
an intracranial monitoring device including a probe;
a cranial plug housing having an intracranial monitoring device recess, the intracranial monitoring device recess is shaped and dimensioned for positioning of an intracranial monitoring device and includes a central access hole shaped and dimensioned for the passage of the probe of the intracranial monitoring device therethrough to a desired position within the brain.

2. The cranial plug according to claim 1, wherein the intracranial monitoring device is a wireless intracranial monitoring device.

3. The cranial plug according to claim 1, wherein the cranial plug housing includes a first end, a second end, a first lateral side, a second lateral side, an upper surface, and a lower surface.

4. The cranial plug according to claim 3, wherein a recessed surface defining the intracranial monitoring device recess is formed along the upper surface of the cranial plug housing.

5. The cranial plug according to claim 3, wherein the lower surface is provided with a projection shaped and dimensioned to fit within the cranial hole.

6. The cranial plug according to claim 3, wherein the intracranial monitoring device recess is formed within the upper surface of the cranial plug housing.

7. The cranial plug according to claim 1, wherein the intracranial monitoring device recess is shaped and dimensioned for receiving the head of the intracranial monitoring device.

8. The cranial plug according to claim 7, wherein the central access hole extends from the recess to the lower surface of the cranial plug housing.

9. The cranial plug according to claim 1, wherein the cranial plug housing includes multiple chemically distinct materials.

10. The cranial plug according to claim 9, wherein the cranial plug housing includes a central body member made of a first material and a second material.

11. The cranial plug according to claim 10, wherein the first material is a sonolucent material.

12. The cranial plug according to claim 11, wherein the first material is PMMA or cubic zirconium.

13. The cranial plug according to claim 11, wherein the second material is a flexible material.

14. The cranial plug according to claim 13, wherein the first material is PMMA or cubic zirconium and the second material is porous polyethylene or expanded polytetrafluoroethylene (EPTFE).

15. The cranial plug according to claim 10, wherein the second material is a flexible material.

16. The cranial plug according to claim 15, wherein the second material is porous polyethylene or expanded polytetrafluoroethylene (EPTFE).

17. A method for installing a cranial plug, the cranial plug including an intracranial monitoring device including a probe and a cranial plug housing having an intracranial monitoring device recess, the intracranial monitoring device recess is shaped and dimensioned for positioning of an intracranial monitoring device therein and includes a central access hole shaped and dimensioned for the passage of a probe of the intracranial monitoring device therethrough to a desired position, the method comprising:
generating virtual images of the cranial plug and the intracranial monitoring device;
upon initiation of a surgical procedure, monitoring in real-time, via a computer based guidance system, the movement of the cranial plug housing and the intracranial monitoring device relative to the patient, wherein tracking devices are integrated into or onto the cranial plug housing and the intracranial monitoring device;
positioning the cranial plug within the patient.
